(19) United States
(12) Patent Application Publication
(10) Pub. No.: US 2006/0293636 A1
(43) Pub. Date: Dec. 28, 2006

(54) Disposable absorbent breathing article

(76) Inventor: David Ocejo-Sanchez, Toluca Lake, CA (US)

Correspondence Address: David Ocejo Sanchez;Suite 142, 10061 Riverside Drive, Toluca Lake, CA, 91602, US

(21) Appl. No.: 11/351,981

(22) Filed: Feb. 10, 2006

(30) Foreign Application Priority Data
Jun. 27, 2005 (MX) PA/A/2005/006997

(51) Int. Cl. A61F 13/15

(52) U.S. Cl. 604/385.22; 604/370

(57) ABSTRACT

An absorbent, disposable and breathing article is described comprising: (a) an upper permeable cloth receiving the discharges of liquid exudates; (b) an absorbent core which retains said liquid exudates; and (c) an impervious backing cloth comprising: a non-woven cloth having an external face which faces toward the user's garment and one internal face which looks toward the absorbent core, and one covering of an extrudable polymeric material, laid on the internal face of said backing cloth, forming a layer impervious to liquids, on said backing cloth.

### CROSS REFERENCE TO A RELATED APPLICATION

This application claims priority to Mexican Patent Application Serial No. PA/a/2005/006997 filed on Jun. 27, 2005 entitled, “DISPOSABLE ABSORBENT BREATHING ARTICLE” in the Spanish language, herein incorporated by reference in its entirety.

### BACKGROUND OF THE INVENTION

### Field of the Invention

A variety of disposable absorbent items are known in the market, such as diapers, absorbent items for incontinent adults and sanitary towels for women.

Said articles generally comprise an upper permeable cloth, an absorbent core and an impervious backing cloth. The upper permeable cloth contacts the user's body, while the impervious backing cloth contacts user's garments. The absorbent core comprises a material capable of absorbing liquid body secretions.

Also, it is generally known that the human body surface generates a skin pH variation, this change favoring the development of skin scuffing or excoriations.

To resolve the problem of scuffing, disposable sanitary products have been suggested, of the so-called breathing type. In said items, the backing cloth is manufactured from materials permeable to vapors, but impervious to liquids. Generally, said materials consist of porous media, however these media are not fully efficient in retaining the absorbent core's liquids, being prone to runoffs at the surface of said cloths.

To deal with the problem, it is proposed giving breathability to diaper, while maintaining an adequate contention for liquids at the backing cloth; to this end several developments have been proposed.

In a first development, it has been proposed a backing cloth comprising a very thin low-density polyethylene belt, laminated over a non-woven hydrophobic spun-bound cloth. A second group includes a backing cloth comprised of a very thin low-density polyethylene belt, glued with adhesive on a non-woven hydrophobic spun-bound cloth. Generally, the polyethylene belt is placed at the diaper's center, at the upper cloth face oriented to the absorbent core.

Through the use of a backing cloth manufactured from a non-woven spun-bound hydrophobic cloth, having an adhered or laminated polyethylene film, it is possible to provide breathability at the diaper's edges, while the diaper's center is held impervious because of the polyethylene belt. Also, with the use of a backing cloth manufactured from a non-woven spun-bond hydrophobic cloth, an appearance and texture similar or alike to that of a fabric is provided to the diaper.

Other developments consist in using two or more non-woven spun-bond hydrophobic clothes of different density as diaper's backing cloth, with absorbent materials provided between both clothes.

The objective of present invention is to provide an improved absorbent item comprising a backing cloth that is breathing and impervious to liquids.

### SUMMARY

### General Background and State of the Art

An objective of the present invention consists in providing a non-woven cloth that is permeable to vapors and impervious to liquids.

A second objective of this invention consists in providing a method for manufacturing a non-woven cloth that is permeable to vapors and impervious to liquids.

Another objective of present invention is to provide an improved disposable item comprised of one non-woven cloth, permeable to vapors and impervious to liquids.

Still another objective of this invention consists in providing an improved breathing disposable diaper and one method for its manufacture.

The abovementioned objectives are reached through providing an improved absorbent article comprised of a breathing backing cloth comprised of a non-woven hydrophobic cloth having a layer formed by means of a bath or coat of polymeric material, extrudable at the central portion of the non-woven hydrophobic cloth.

### BRIEF DESCRIPTION OF THE DRAWINGS

FIG. 1 shows an upper view of the absorbent item, subject of present invention.

FIG. 2 shows a cross section along line A-A′, of a first embodiment of an absorbent article, subject of present invention.

FIG. 3 shows a cross section along line A-A′, of a second embodiment of an absorbent article, subject of present invention, without barriers.

### DETAILED DESCRIPTION OF THE PREFERRED EMBODIMENTS

According to present invention, a disposable absorbent breathing article is described. The term “disposable absorbent article” refers to an item that absorbs and restrains body exudates, for example urine, fecal substances and menstrual flows, where said item will not be washed to be reused, but will be disposed of.

Examples of disposable absorbent articles comprise disposable diapers, training diapers, diapers for incontinent adults, sanitary towels, incontinence pads and pantiliners (linings for panties).

Present invention is described with reference to a disposable diaper, but as will be evident to those skilled in the art, the invention is not limited to a disposable diaper, but can be applied to any of the abovementioned disposable absorbent articles.

Clothes used for the absorbent items of present invention are non-woven hydrophobic clothes. These clothes can be treated with surfactants in order to partially acquire the hydrophilic property; then these treated non-woven clothes are said to be “hydrophilic”. Actually, these clothes remain hydrophobic, only requiring less pressure in order liquids can get through said cloth, compared to pressures require if it had no surfactant treatment. In present invention, “hydrophilic” or “permeable” cloth will be used for non-hydrophobic clothes, which have received a treatment with surfactant, and which allow passage of liquids through them. In contrast, non-woven “hydrophobic” cloth refers to a non-woven cloth without surfactant treatment, which does not allow passage of liquids through it. Also, according to present invention, by “impervious” non-woven cloth will be indicated a non-woven cloth which has a layer of extruded polymer.

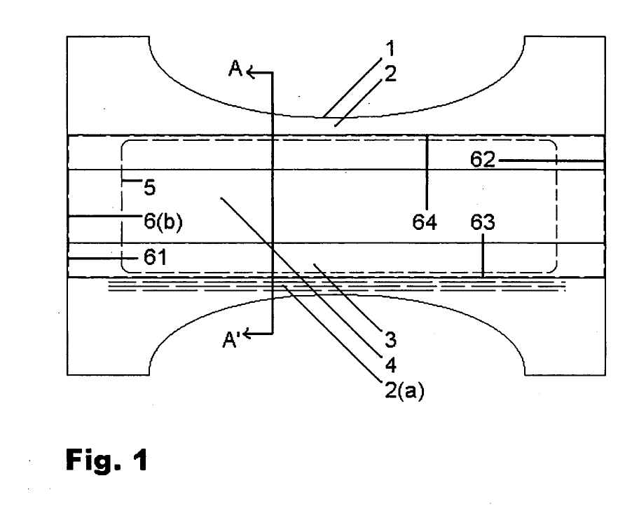

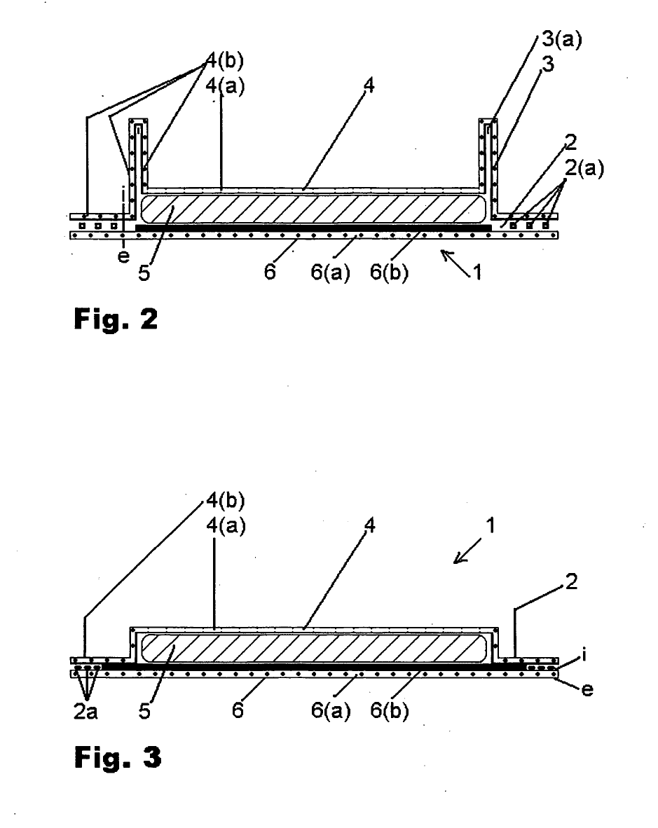

According to FIGS. 1-3, with number 1 is designated the absorbent article of the invention, being illustrated as a disposable absorbent diaper. With number 2 is indicated the leg flap of diaper, number 2(a) indicates the elastic wilt of the leg flap. Number 3 indicates an elasticized barrier. Number 3(a) shows the barrier welt 3. Number 4 indicates the non-woven upper cloth, possessing a central “hydrophilic” or “permeable” portion 4(a) and lateral “hydrophobic” portions 4(b). The central “hydrophilic” portion 4(a) corresponds to the area where liquid exudates are discharged, while the “impervious” portions 4(b) correspond to the material which forms barriers 3 and leg flaps 2. The leg flap 2 is formed with a portion of the upper cloth and a portion of the backing cloth. However, diaper's leg flaps 2 can lack of upper non-woven cloth portion, or that one of the backing cloth. Or plainly, the diaper can have no leg flaps.

The upper and backing clothes which form the leg flap are both non-woven and permeable to vapors, or breathing.

As will be evident to a knowledgeable in the art, the non-woven cloth used as upper and backing cloth can be partially or totally hydrophilic.

Number 5 indicates the absorbent core of the disposable sanitary product. With number 6, the non-woven backing cloth is shown. Backing cloth 6 comprises a non-woven cloth 6(a) and a layer 6(b) of an extrudable polymer. The non-woven cloth 6(a) has an outer face e, oriented, when in use, towards the user's garment, and an inner face i oriented towards the absorbent core 5.

The upper cloth 4 comprises a portion contacting the user's skin, and although manufactures with an hydrophobic material, is “permeable” or “hydrophilic” to liquids, by virtue of a treatment with surfactant agents.

The non woven cloth that can be used as upper cloth 4 comprises a fibrous cloth, manufactured using a spun bond or carding process. Preferred non-woven materials are manufactured from synthetic fibers, comprising polyethylene, polypropylene, polyester and cellulose. Particularly preferred are non-woven materials manufactured from polyester, polyethylene or polypropylene fibers, or mixtures of these.

The use of non-woven hydrophobic permeable materials, by a treatment with surfactant agents, is described in U.S. Pat. No. 3,838,6924 of Levesque y U.S. Pat. No. 4,073,852 of Mesek.

Preferably, the upper cloth can be formed from a unique cloth containing surfactant treatment in at least the central zone of the diaper, corresponding to the liquid exudates' discharge, causing the center of the diaper being permeable, and to form the leg flaps with the edge hydrophobic untreated portions. The building of said upper cloth is shown in Mexican Patent No. 191,385 of David Ocejo Sanchez.

In another embodiment, the upper cloth consists of a non-woven cloth having parts of the leg flap with or without surfactant treatment, comprising also resilient flaps manufactured from a non-hydrophobic non-woven cloth, adhered along the diaper, in order to contact the user's crotch. The building of said upper cloth is shown in Mexican Patent No. 161,320, of Lawson.

Another embodiment consists of an “hydrophilic” non-woven central cloth, to which two hydrophobic “impervious” clothes are adhered in two opposite edges, and from which the leg flap and the elasticized barrier are formed. The building of said upper cloth is described in Mexican Patent 174,128 of Dragoo, and in Mexican Patent No. 182,636 of Huffman.

Although diapers with elasticized barriers are preferred, the absorbent items of present invention can lack of barriers 3. In FIG. 3, an absorbent item without elasticized barriers is shown.

In the case of diapers, the leg flaps 2 are formed from the non-woven cloth material at the edges of the backing- and upper-clothes.

The absorbent core 5 can be any absorbent material capable of absorbing and retaining liquids as urine or menstrual flows. The absorbent core 5 can be manufactured in several forms and dimensions; for example, rectangular, in the form of a hourglass, asymmetrical, in the form of a bone, etc. For manufacturing the absorbent core, materials that can be used include shredded woodpulp, napped cellulose, synthetic fibers, tissue paper, absorbent sponges, superabsorbent polymeric materials, gellant materials, and combinations of them. In a preferred embodiment, the absorbent core 5 comprises shredded woodpulp and superabsorbent polymers, located in the portions for liquids' discharges.

The absorbent core 5 optionally comprises a tissue paper envelope, and optional can comprise a distributing layer. The former types of absorbent cores are well known in the art.

According to present invention, the material used as backing cloth 6 of the absorbent article consists of a non-woven hydrophobic or hydrophilic cloth, preferably hydrophobic 6(a), having a bath or cover 6(b), of extrudable polymer.

The nonwoven cloth that can be used as backing cloth 6(a) comprises a fibrous cloth, manufactured using a spun bond or carding process. Preferred non-woven materials are manufactured from synthetic fibers, comprising polyethylene, polypropylene, polyester and cellulose. Particularly preferred are non-woven materials manufactured from polyester, polyethylene or polypropylene fibers, or mixtures of these.

The density of the material used as backing cloth 6(a) is between 5 and 200 g/m2, preferably from 10 to 60 g/m2. The non-woven cloth used as backing cloth comprises an external face e which faces toward the user's garments, and an internal face i, facing towards the absorbing core 5.

According to present invention, the hydrophobic and impervious cloth's 6(a) internal face i gets a cover of an extrudable polymer, in such a way that a bath or covering 6(b) be formed in all or part of the cloth's surface 6(a). The cover 6(b) is deposited at the inner face i of the non-woven cloth 6(a) facing towards the absorbent core, while the external face e, without covering, looks toward the user's garment, while in use.

The extrudable polymer wets the fibers and covers the pores of the non-woven cloth. To this end, a curtain of extrudable polymer is formed, of a certain thickness, which baths a non-woven cloth. Alternatively, the extrudable polymer can be injected through the non-woven cloth in such a manner that it penetrates the non-woven cloth's structure, sealing the fiber pore and the inter-fiber pores, bathing also the polypropylene fibers of said cloth, therefore providing an impervious nature for said clothes.

The bath or covering 6(b) of extrudable polymer is flexible, and it does not fracture nor cracks because of a flexure caused by the user's movement during its use.

According to present invention, the extrudable polymer cover or bath is applied only to the body exudates' discharge area. That is, the central area extending from approximately the longitudinal edges 61 and 62, which form the waist of the diaper, and the lateral edges 63 and 64, along the line where the leg and diapers' flaps are defined, and where appropriate, the base of diaper's barriers. Although the leg flaps can be formed from portions of the upper and backing clothes, said leg flaps are not considered as forming part of the exudates' discharge zone.

Nevertheless, as will be evident to a knowledgeable in the technique, the bath or covering can extend a little beyond the base of the barriers (lines 63, 64), and also variations can exist, derived from the manufacturing tolerances for the equipment used. Even more, the bath or covering can extend up to the zone adjacent to the elastics which form the leg flap, as shown in FIG. 3. Or, the covering can extend to the whole cloth 6(a).

As already indicated, the covering 6(b) can extend longitudinally to the whole length of the diaper. Alternatively, the covering can extend only to approximately 1 cm beyond the absorbent core area.

For locating the covering in the backing cloth, in the exudates' discharge area, the avoidance has to be foreseen of allowing the runoff of exudates through the backing cloth, discharged and retained into the absorbent core.

Also, the manufacturing adhesive for the absorbent article avoids the leakage of liquids at the lateral or longitudinal edges of the diaper, out of the covering area.

The covering thickness 6(b) of extrudable polymer is in the range between 0.1 to 0.9 mm.

Preferred materials to use as extrudable polymers are the so-called hot-melt polymers, for example polystyrene copolymers, polystyrene-poly(ethylene-butylene)-polystyrene copolymers, and polystyrene-polyisoprene-polystyrene copolymers. Nevertheless, as will be evident to a knowledgeable in the art, also polymers cold-formable can be also used.

The extrudable polymer covering forms a polymeric impervious layer 6(b) that seals the pores of the non-woven substrate structure 6(a), said polymeric covering being impervious to liquids.

The mass-manufacture of the absorbent articles according to present invention comprises:

a) Providing an upper non-woven “permeable” cloth,

b) Providing an absorbent core,

c) Providing a backing non-woven cloth, covered with an extrudable polymer, and

d) Assembling an absorbing article.

Example of a Preferred Execution.

The diaper for present invention is manufactured at a production line for diapers or similar absorbent products. The upper permeable cloth of present invention is obtained from a coil of non-woven cloth having a center treated with surfactant agents, in order to allow the permeance of the liquid through said material. While it has lateral edges that hold their hydrophobic character. In other embodiment, the upper cloth can be totally made of an hydrophilic material.

In the case of diapers with barrier, according to present invention, near each edge of upper cloth, barriers are formed, as well as leg flaps in the hydrophobic portion of the non-woven cloth.

Barriers can be of the double-layer type, where the barrier comprises two hydrophobic material barriers; or also, the barriers can be of the monolayer type. In this configuration, barriers are adhered to the upper cloth' edges, in such a way that the pervious portion of upper cloth, at the barrier, ends at approximately the base of said barriers.

The manufacture of the absorbing core comprises a step of formation of the absorbent material, a second step of inclusion of super absorbent polymers, which are attached to the absorbing material by means of adhesive, and optionally, a stage of wrapping into tissue paper.

The manufacture of the backing cloth comprises bathing a non-woven cloth with an extrudable polymer, in order to form a polymeric layer on the cloth.

Differently to the material used as upper cloth, the non-woven cloth for the backing cloth does not comprise a treatment with surfactants for reducing its imperviousness to liquids. Alternatively, the material used as backing cloth can previously receive a treatment with surfactants, for augmenting its impervious properties. These treatments include, for example, treatment with silicon-based polymers, or siliconed polymers, or any treatment with surfactant agents, for incrementing its hydrophobic character.

In the step of bath or covering, an extrudable polymer, preferably a hotmelt, is applied on the inner face of backing cloth, adjacent to the absorbing core. The application of the hot-melt polymer is done through an extruding die having a slit and shims which help the formation of a polymer curtain of a certain thickness, bathing the surface being covered, and forming a polymeric layer on the non-woven cloth, having the predetermined thickness.

The extruding die for the extrudable polymer bath is preferably located at the diapers- or similar absorbent articles' production line, before the positioning of the absorbent core.

In case a hotmelt polymer is used, the hotmelt viscosity, while covering the internal face of backing cloth, is in the range of some 1,000 to 1,500 cps. To this end, the temperature of the hot-melt polymer is between approximately 120 and 170° C. The hot-melt polymer mass is in the approximate range from 5 to 25 g/m2. It has been found that the layers formed using masses below 5 g/m2 are not efficient for providing an impervious cloth having properly sealed pores.

Also, it is possible to form the extrudable polymer covering through injecting said polymer to the non-woven cloth, thus sealing the pores into/and between the fibers in all the thickness of the hydrophobic or hydrophilic non-woven cloth, in order to provide its imperviousness.

In the case of disposable diapers, before gathering the elements forming the disposable absorbent article, the elastic components forming the elasticized leg flaps and the elastic belts forming the waist wand are put, as well as, if applicable, the attaching means for the diaper, such as adhesive tabs and reinforcements for the diaper's front; or else, the mechanical attachment media are put, such as belts of loops and grabs of the type known as velcro.

The curing of the covering is allowed, and promptly the assembly of the backing, upper and absorbent core clothes are done to manufacture the disposable absorbing article.

Although the invention has been described with reference to the drawings, these are not limitations for the invention, and the scope of the invention shall be considered according to following Claims.

I claim:

1. An absorbent, disposable and breathing article comprising:
an upper permeable layer, to receive the liquid exudates' discharges an absorbent core, to contain said liquid exudates; and
an impervious backing cloth, comprising:
a non-woven cloth having an external face, looking toward the user's garment and an internal face, looking toward the absorbent core; and
a covering made of an extrudable polymeric material, on the internal face of said backing cloth, that forms a layer impervious to liquids at said backing cloth.

2. The absorbent article according to claim 1, whereupon said extrudable polymeric material covering extends in the area where body exudates are discharged.

3. The absorbent article according to claim 2, whereupon said extrudable polymeric material covering extends from approximately one end to the other longitudinal end forming the waist of the diaper, and from approximately one edge to the other edge defining the base of the lateral barriers and the leg flaps.

4. The absorbent article according to claim 1, whereupon said covering extends to the whole length of the diaper, in longitudinal direction.

5. The absorbent article according to claim 1, whereupon said extrudable polymeric material is a hot-melt polymeric material.

6. The absorbent article according to claim 1, whereupon said extrudable polymeric material is a polymeric material extrudable in cold.

7. The absorbent article according to claim 1 whereupon the polymeric film has a density of 5 to 25 g/m².

8. The absorbent article according to claim 1, whereupon the polymeric film consists of a bath applied through an extrusion die.

9. The absorbent article according to claim 1, whereupon the polymeric film is injected into the non-woven cloth, sealing pores and bathing the fibers of said non-woven cloth of the said backup cloth.

10. A procedure for the manufacture of a diaper, comprising: (a) providing an upper cloth; (b) providing an absorbent core, (c) providing a non-woven backup cloth, (d) in-line covering said backup cloth with an extrudable polymer and (e) assembling an absorbent article.

11. The procedure for manufacturing a diaper according to claim 10, whereupon the step of in-line covering said backup cloth with an extrudable polymer comprises bathing said backing non-woven cloth with an extrudable polymer by means of an extruding die.

12. The procedure for manufacturing a diaper according to claim 10, whereupon the step of in-line covering said backup cloth with an extrudable polymer comprises injecting an extrudable polymer into the backing non-woven cloth.

13. The procedure for manufacturing a diaper according to claim 10, whereupon said polymeric extrudable polymer comprises a hot-melt polymeric material, which has a viscosity in the approximate range from 1,000 to 1,500 cps and is at a temperature in the approximate range from 120 to 170° C.

14. The procedure for manufacturing a diaper according to claim 10, whereupon said polymeric extrudable material is a polymeric material cold-extrudable.

15. A backing cloth to be used in an absorbent article, comprising a non-woven cloth having an external face looking towards the user's garment, and an internal face looking toward the absorbent core, characterized because the face looking towards the absorbent core comprises a bath of a polymeric extrudable material, applied to said face, and forming a layer impervious to liquids, on the inner face of said cloth.

16. The backing cloth to be employed in an absorbent article according to claim 15, whereupon said extrudable polymeric material is a hot-melt polymeric material.

17. A procedure for preparing a backing cloth for an absorbent article, which comprises providing to at least one face of a non-woven cloth, a covering or bath of one extrudable polymer, with the purpose of providing imperviousness to it.

18. A procedure for manufacturing a backing cloth to an absorbent article according to claim 17, whereupon said extrudable polymer comprises a hot-melt polymeric material, which has a viscosity in the approximate range from 1,000 to 1,500 cps and is at a temperature in the approximate range from 120 to 170° C.
